(19) United States
(12) Patent Application Publication
(10) Pub. No.: US 2011/0111063 A1
(43) Pub. Date: May 12, 2011

(54) Extended Moisturization Cosmetic Compositions

(76) Inventor: Akira Yokozeki, New York, NY (US)

(21) Appl. No.: 12/768,825

(22) Filed: Apr. 28, 2010

Related U.S. Application Data
(60) Provisional application No. 61/176,970, filed on May 11, 2009.

(51) Int. Cl. A61K 8/97; A61Q 19/00

(52) U.S. Cl. 424/725

(57) ABSTRACT

A cosmetic composition providing a skin moisturizing effect over extended periods of time when applied to skin in need thereof is provided. The composition comprises a skin moisturizing sufficient amount of an extract of an air plant in combination with a cosmetically or dermatologically acceptable carrier. Also provided is a method for providing a skin moisturizing effect over extended periods of time. The method comprises applying to the skin in need of a skin moisturizing effect, a composition comprising a skin moisturizing sufficient amount of an extract of air plant in combination with a cosmetically or dermatologically acceptable carrier.

### CROSS-REFERENCE TO RELATED APPLICATIONS

The present application claims priority of U.S. Provisional Application No. 61/176,970 filed on May 11, 2009.

### FIELD OF THE INVENTION

The present invention relates to cosmetic formulations for topical application to the skin. More particularly, the present invention concerns skin care compositions, and more specifically skin care compositions which continually condition, hydrate and moisturize the skin, maintaining the skin's moisture balance all day long while the product is worn.

### BACKGROUND OF THE INVENTION

Tight, smooth skin contributes to a youthful and attractive appearance. Skin creases and wrinkles, caused by sun damage, smoking and aging, make people look older. Although injectable soft tissue skin and wrinkle fillers, such as fat, collagen and hyaluronic acid, can be used to increase tissue volume, reduce wrinkles, and to therefore improve the overall smooth and tight appearance of skin, many consumers do not wish to incur the expense and the discomforts of using this skin tightening regimen. Moreover, as nothing stops the skin from aging, these treatments need to be repeated to maintain benefits.

Other skin treatments include the use of medications, such as topical retinoids; surgical procedures, such as dermabrasion, microdermabrasion, laser, chemical peel, Botox, and facelifts. Many consumers also rely on nonprescription or cosmetic wrinkle creams. The effectiveness of such creams relies on the active ingredient, such as retinol, hydroxy acids, peptides, antioxidants, and the like. Since cosmetic products contain lower concentrations of these active ingredients than do prescription formulations, actual improvements observed in the depth of wrinkles and the smoothness of the skin are likely to be modest.

Many consumers also rely on their makeup products, for example, foundations or powders, to reduce at least the appearance of lines and wrinkles to create a smoother and more youthful look. Many of these makeup products include actives which are claimed to provide long-term benefits with continued use. Nevertheless, cosmetic powders, in particular, have not previously been knows to condition and hydrate the skin and maintain the skin's moisture at a comfortable level. In fact, powders, when applied to the skin, typically are felt by consumers as if they are absorbing skin's moisture along with sebum, since the powder causes the skin to feel tight and mask-like on application. Over time, the skin is prone to dry out which facilitates the development of lines and wrinkles. It would be desirable to provide a cosmetic powder, such as in the form of a foundation, which continually conditions, for example, which protects, hydrates and/or moisturizes, the skin so as to delay the onset of wrinkles and maintain the skin's youthful appearance. It would also be desirable to provide a skin conditioning powder which feels protecting, hydrating and/or moisturizing immediately upon application and over time. It would further be desirable to provide a skin conditioning powder which leaves skin feeling smooth, supple and rejuvenated immediately upon application and over time. It would also be desirable to provide a skin conditioning powder which leaves skin feeling firmer, but not mask-like, immediately and over time. It would be desirable to achieve these results using natural ingredients. The present invention achieves these desired results by incorporating into a powder composition a skin conditioning sufficient amount of an extract of an epiphyte, sometimes called “air plant” to speed moisturization to the skin.

Epiphytes are organisms which grow upon or are attached to a living plant or host. Epiphytes or epiphatic plants are among the most fascinating of all plants because they do not root in soil. These organisms derive only physical support and not nutrition from their host. Epiphytes use photosynthesis for energy, and the non-aquatic forms obtain moisture from the air (i.e., humidity from rain or cloud moisture) on the surface of their hosts. Roots may develop primarily for attachment to the host, and specialized structures, such as cups and scales, may be used to collect or hold moisture. Nutrition is derived from rain water nutrients and decomposing matter (mostly leaves and dead insects) that may be caught among their roots or in their leaves by structures called trichomes. Additionally, lightening produces nitrogen in the air that epiphytes absorb. The best known epiphatic plants include mosses, orchids, and bromeliads such as Spanish moss (of the genus Tillandsia). Common names for Tillandsia include air plant, ball moss (T. recurvata) and Spanish moss, the latter referring to T. usneoides in particular. It has now been discovered that a skincare composition according to the present invention, which incorporates an extract of air plant, delivers substantially immediate and continuous conditioning to the skin, maintaining the skin's moisture balance all day while the foundation is worn.

### SUMMARY OF THE INVENTION

An object of the instant invention is to provide a cosmetic composition which will provide a skin conditioning effect when applied to the skin.

Another object of the instant invention is to provide a skin conditioning cosmetic composition which will provide a skin conditioning effect over extended periods of time when applied to the skin.

A further object of the instant invention is to provide a skin conditioning cosmetic powder composition which will provide a skin conditioning effect over extended periods of time when applied to the skin.

Yet a further object of the instant invention is to provide a method for conditioning the skin over extended periods of time comprising applying to the skin in need of extended skin conditioning a skin conditioning cosmetic powder composition.

These, and other objects that will be made apparent in the detailed description to follow, are accomplished according to the present invention by providing a skin conditioning composition comprising a skin conditioning sufficient amount of an extract of an air plant in a cosmetically or dermatologically acceptable carrier.

### BRIEF DESCRIPTION OF THE DRAWINGS

FIG. 1 is a graph illustrating the level of moisturization achieved on skin using a powder foundation composition according to the present invention compared with a commercial product in which each of the compositions is applied to skin with a dry nylon sponge.

FIG. 2 is a graph illustrating the level of moisturization achieved on skin after application to skin of a powder foundation composition according to the present invention compared with a commercial product in which each of the compositions is applied to skin with a water-dampened nylon sponge.

### DETAILED DESCRIPTION OF THE INVENTION AND PREFERRED EMBODIMENTS THEREOF

Reference will now be made in detail to preferred embodiments of the invention.

Skin is an organ composed of multiple layers of tissue, the most external layer of which is the stratum corneum. A basal layer underlies the stratum corneum, and the dermis underlies the basal layer. The skin protects the body and serves as a moisture reservoir. The stratum corneum, composed of keratinized cells, polysaccharides and lipids, functions as a moisture barrier, retaining moisture in the skin. Water is critical to maintaining the proper condition and appearance of the skin. The amount of water in the skin depends on the skin's ability to bind water, the rate at which water is supplied from the internal skin layers and the rate at which water is lost due to evaporation. Incorporating water in various cosmetically acceptable bases, containing skin conditioning agents, improves the availability of water to the skin and water take-up by the skin, when the cosmetic compositions are applied to the skin. Skin conditioning agents include emollients which lubricate the skin surface and gives skin a soft and smooth appearance; skin protectants which temporarily protect injured or exposed skin from harmful or annoying stimuli and which may provide relief to such skin; occlusives which slow the loss of water from the skin by forming barrier on skin's surface; humectants which increase the water content of the top layers of skin by drawing moisture from the surrounding air and hold moisture in the skin, and miscellaneous skin conditioning agents which enhance the appearance of dry or damaged skin by reducing flaking and restoring suppleness.

Typically, skin conditioning ingredients in cosmetic products function in direct relation to the amount of the product applied. However, cosmetic products according to the present invention, containing a skin moisturizing sufficient amount of an air plant extract, are capable of delivering unlimited, continuous hydration to the skin, maintaining the skin's moisture balance all day as long as the product is worn. While not wishing to be bound by any particular theory, it is believed that the extract of the air plant functions as a natural skin conditioning agent having one or more of the properties of conventional skin conditioning materials, that is, emollient, skin protectant, humectant, and so forth, to lubricate, protect the skin, improve the appearance of, and/or draw moisture to, and retain moisture in the skin, all day, while the product is being worn.

For purposes of illustration only, the present invention will be described in the form of powder cosmetic, for example, an anhydrous or substantially anhydrous powder composition, such as a powder foundation, compressed or loose powder, blush or eyeshadow, for topical application to the facial skin. By “anhydrous” or “substantially anhydrous”, it is meant that such powders may include up to about 1.0 percent water by weight of the total composition, including any amount of water in between 0 and 1.0 percent. Preferably, if water is present in the composition, it is present in an amount of from about 0.01 to about 0.8 percent, and more preferably from about 0.1 to about 0.5 percent, such as about 0.4 percent water. In a preferred embodiment of the invention, the composition is a powder foundation comprising a skin conditioning sufficient amount of an extract of air plant in a powder vehicle.

The amount of the extract of air plant useful in the compositions of the instant invention, whether in the powder or other formulation, generally is in the range of from about 0.001 to about 10 percent by weight of the total composition, including any amount in-between. Preferably, the extract of air plant is present in the compositions in an amount in the range of 0.01 to about 5 percent by weight of the total composition. More preferably, the amount is in the range of from about 0.2 to about 2 percent by weight of the total composition, such as in the range of from about 0.2 to about 1, for example, about 0.4 percent, by weight of the total composition. Preferably, the extract of air plant is derived from T. usneoides.

The powder component is present in the composition in an amount in the range of about 80 to about 99 percent by weight of the total composition, including any amount in-between, and more preferably, in the range of from about 85 to about 95 percent by weight of the total composition, such as about 85 percent by weight of the total composition. Suitable powders for use in the compositions of the present invention include mica and titanium dioxide. The layers of mica particles reflect light and create an illusion of smoother, softer and more radiant skin. Fine lines are softened and skin texture appears more even. Mica is also sheer and translucent with a natural gloss which allows the user's natural skin tone to shine through. Titanium dioxide is a natural pigment which also functions as a physical sunscreen.

The powder component may also include one or more colorants, for example, pigments, such as iron oxides. If present, the colorants may be present in the composition in an amount in the range of from about 0.01 to about 30 percent, including any amount in-between, preferably, in an amount of from about 0.1 to about 20 percent, and more preferably, in an amount in the range of from about 1 to about 10 percent, such as an amount in the range of from about 1.5 to about 5 percent, such as about 2 percent, by weight of the total composition.

The powder foundation compositions of the present invention may also contain soft focus powders, such as nylon 12, bismuth oxychloride, methicone, dimethicone, and the like; slip modifiers, such as zinc myristate, caprylic/capric triglyceride, dimethicone/vinyl dimethicone crosspolymer, and the like; opacifying agents, such as aluminum hydroxide; mattifying agents, such as silica; surfactants or emulsifiers, such as sorbitan sesquiisostearate.

The compositions of the present invention, whether in the powder formulation or another form, may further include one or more additional skin conditioning agents, including, emollients, skin protectants, occlusives, miscellaneous skin conditioning agents, and humectants. Exemplary moisturizing, emollient or skin conditioning agents include, but are not limited to, caprylic/capric triglyceride, C9-15 fluoroalcohol phosphate, isotridecyl isononanoate, hyaluronic acid, glycerin, urea, trehalose, and the like. Non-limiting examples of humectants useful in the compositions of the present invention include glycerin, ethylhexylglycerin, pentylene glycol, butylene glycol, propylene glycol, sorbitol, hyaluronic acid, urea, lactic acid, and the like. In a preferred embodiment of the present invention, glycerin is provided in an encapsulated form to aid in the distribution of glycerin throughout the powder formulation. In a particularly preferred embodiment of the present invention, the capsule is a silk protein and silicone oil-containing capsule, such as where the silk protein component is trimethylsilyl hydrolyzed silk PG-propylmethylsilanediol crosspolymer. When the powder composition, containing the encapsulated glycerin is applied to the skin, the capsules break open and distribute silk peptides and glycerin homogeneously throughout the composition. When used in the powder compositions of the instant invention, the encapsulated humectant will be present in an amount in the range of from about 0.06 to about 15%, including any amount in-between, for example, from about 0.06 to about 7.5%. In one embodiment of the instant invention, the encapsulated humectant (i.e., the total weight of the humectant in combination with the capsular material) is present in an amount of about 1.3% by total weight of the composition. In another embodiment of the instant invention, the encapsulated humectant is present in an amount of about 6.5% by total weight of the composition.

In a powder formulation according to the present invention, the additional skin conditioning agents may be present in an amount in the range of from about 0.0001 to about 15 percent by weight of the total composition, including all amounts in-between, such as from about 0.01 to about 10 weight percent, for example, about 0.1 to about 5 weight percent. When the composition is in the form of a lotion, a cream, a cleanser, or the like, the additional skin conditioning agents may be present in the compositions of the present invention in an amount of from about 0.001 to about 60 percent, including all amounts in-between, such as from about 0.5 to about 50 percent, for example, from about 10 to about 40 percent by weight of the total composition.

Oils and oil-related ingredients, such as silicones, may be present in the instant compositions in an amount in the range of from about 1-90%, including any amount in-between, such as an amount in the range of from about 65 to about 80%. In a powder formulation according to the instant invention, the amount of oils and oil-related ingredients may be present in the range of from about 1 to about 20 weight percent, including all amounts in-between, such as in the range of about 3 to about 15 weight percent, for example, about 6 weight percent, by total weight of the composition.

Compositions of the present invention, whether in the powder formulation or otherwise formulated, may also contain other cosmetic ingredients, such as vitamins, collagen synthesis or blood circulation enhancing agents, anti-oxidants, plumping agents, anti-wrinkle or skin tightening agents, sunscreens, anti-aging agents, skin whitening or depigmentation agents, anti-inflammatory agents, anti-acne agents, DNA repair agents, pore minimizing agents, and the like. Exemplary vitamins include Vitamins C and E. Exemplary collagen synthesis or blood circulation enhancing agents include, but are not limited to, arginine, Ascophyllum nodosum extract, Asparagopsis armata extract, caffeine, and the like. Exemplary anti-oxidants include, but are not limited to, nordihydroguaiaretic acid, grape seed extract, green tea leaf extract, and the like. Exemplary plumping agents include, but are not limited to, Saccharomyces/xylinum black tea ferment, Anemarrhena asphodeloides root extract, sodium hyaluronate, and the like. Exemplary anti-wrinkle agents include, but are not limited to, acetyl hexapeptide-8, palmitoyl oligopeptide, dipeptide diaminobutyroyl, benzylamide diacetate, and the like. Exemplary skin-tightening agents include, but are not limited to, algae extract, pullulan, sweet almond seed extract, carbomer, palmitoyl oligopeptide, palmitoyl tetrapeptide-7, Quercus suber extract, and the like. Exemplary sunscreens include ethylhexyl methoxycinnamate. Exemplary anti-aging agents include, but are not limited to, teprenone, trisodium resveratrol triphosphate, Polygonum cuspidatum root extract, whey protein, and the like. Exemplary skin-whitening or depigmentation agents include, but are not limited to, ascorbic acid, magnesium ascorbyl phosphate, aminopropyl ascorbyl phosphate, mulberry root extract, Scutellaria baicalensis extract, grape extract, ferulic acid, hinokitol, and the like. Exemplary anti-inflammatory agents include, but are not limited to, spike moss extract, seal whip extract, Polygonum cuspidatum root extract, and the like. Exemplary anti-acne agents include, but are not limited to, salicylic acid, glycolic acid, lactobionic acid, and the like. Exemplary DNA repair agents include, but are not limited to, C1-C8 alkyl tetrahydroxycyclohexanoate, micrococcus lysate, bifida ferment lysate, and the like.

It will be apparent to one of ordinary skill in the art that the present disclosure may be adapted for use with any cosmetic product in addition to powders, e.g. a solubilized lotion, an emulsion, or a solubilized emulsion, such as a lotion, a cream, a cleansing oil or an essence or serum, to be applied to keratinous materials including the skin, including the facial skin, the eyelashes, the eyebrows and the hair.

Although the invention is described in terms of a powder cosmetic, one skilled in the art will appreciate that the present invention may be embodied in lip products, skin and hair care products, and cleansing products.

Although the present invention has been described in accordance with preferred embodiments, it will be understood by those skilled in the art that various changes and modifications may be made without departing from the spirit and scope of the invention.

The invention will be described further by reference to the following non-limiting examples.

### EXAMPLES

### Example 1

### Powder Foundation—(BPK WT-17)

A powder foundation composition according to the present invention is prepared according to the formulation reported in Table 1.

TABLE 1

| | |
|---|---|
| Ingredients | Weight percent |

Phase I

| | |
|---|---|
| Mica | 61.31 |
| Ethylhexyl methoxycinnamate | 6.00 |
| Nylon-12 | 3.00 |
| Zinc myristate | 2.50 |
| Silica | 2.40 |
| Sorbitan sesquiisostearate | 0.20 |
| Isotridecyl isononanoate | 0.08 |
| Iron oxides | 1.70 |

Phase II

| | |
|---|---|
| Titanium dioxide | 15.20 |
| Dimethicone | 1.50 |
| Glycerin | 1.00 |
| Methicone | 0.90 |
| Caprylic/capric triglyceride | 0.70 |
| Pentylene glycol | 0.50 |
| Phenoxyethanol | 0.52 |
| Ethylhexylglycerin | 0.50 |
| Trimethylsilyl hydrolyzed silk | 0.30 |
| PG-propylmethyl silandiol crosspolymer | |
| Aluminum hydroxide | 0.30 |
| Tillandsia usneoides extract (20%) in | 1.00 |
| butylene glycol and water | |
| Tocopherol | 0.20 |
| Dimethicone/vinyldimethicone crosspolymer | 0.10 |
| C9-15 fluoroalcohol phosphate | 0.09 |
| TOTAL | 100.00 |

Procedure: Phase I ingredients are blended. Then, Phase II ingredients are individually added gradually, to the Phase I ingredients, with mixing. When the mixture is homogeneous, it is crushed into small particles.

### Example 2

### Clinical Study

A consumer study was conducted to evaluate the moisturization performance of the powder foundation of Example 1 as compared with the performance of a commercial non-drying powder foundation product, Clinique Derma White powder (DW), the formula for which is provided below in Table 2.

TABLE 2

| Ingredients | Weight Percent |
|---|---|
| Talc | 19.68018 |
| Mica | 18.75235 |
| Synthetic fluorphlogopite | 9.80000 |
| Titanium dioxide | 13.35300 |
| Acrylates/Ethylhexyl acrylate crosspolymer | 6.85650 |
| Ethylhexyl methoxycinnamate | 5.50000 |
| Polymethyl methacrylate | 5.00000 |
| Dimethicone | 4.38700 |
| Zinc oxide | 3.31280 |
| Silica | 3.01800 |
| Iron oxides | 2.06694 |
| Isocetyl myristate | 1.50000 |
| Magnesium myristate | 1.20000 |
| Vinyl dimethicone/methicone | 1.00000 |
| silsesquioxane crosspolymer | |
| C9-15 Fluoroalcohol phosphate | 0.62261 |
| Methicone | 0.57250 |
| Petrolatum | 0.50000 |
| Phenyl trimethicone | 0.50000 |
| Dimethicone/vinyl dimethicone crosspolymer | 0.45000 |
| Caprylyl glycol | 0.27500 |
| Microcrystalline cellulose | 0.25000 |
| Triethoxycaprylylsilane | 0.20000 |
| Aluminum hydroxide | 0.19383 |
| Phenoxyethanol | 0.17500 |
| Lauroyl lysine | 0.16089 |
| Isostearyl sebacate | 0.15000 |
| Silica dimethyl silylate | 0.14350 |
| Timethylsiloxysilicate | 0.11000 |
| Schizosaccharomyces ferment extract filtrate | 0.10000 |
| Hexylene glycol | 0.05000 |
| Stearyl glycyrrhetinate | 0.05000 |
| Disodium stearoyl glutamate | 0.02500 |
| Tocopherol | 0.02000 |
| Hydroxyapatite | 0.01440 |
| Phytosteryl/octyldodecyl lauroyl glutamate | 0.01000 |
| Polyquaternium-61 | 0.00050 |
| TOTAL | 100.00000 |

Three female panelists, between the ages of 25 and 39, participate in the study.

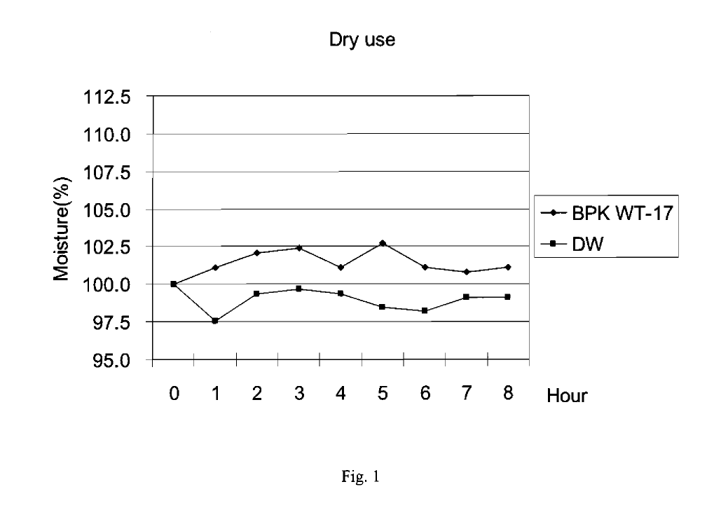

In Part I of the study, each of the panelists self-applies 0.1 gram of the powder foundation of the invention to the left half of the face, and 0.1 gram of the conventional foundation to the right half of the face, with separate nylon sponges. The formulations are applied directly to clean, unmoisturized and unprimed skin. Skin moisturization is tested on the skin of the cheek bones, using MOISTURE CHECKER, model no. MY-707S (Scalar Co. LTD, Japan), each hour for a total of 8 hours. The instrument measures an output proportional to the skin's electrical capacitance in percent. Skin moisturization is measured as a function of increased skin surface water content. The higher the water content of the skin, the higher the electrical capacitance and the more moisturized the skin. The results of Part I are shown in FIG. 1. As shown in the figure, the composition of the present invention, when applied dry, results in greater skin moisturization over the 8 hour period as compared with the conventional powder foundation.

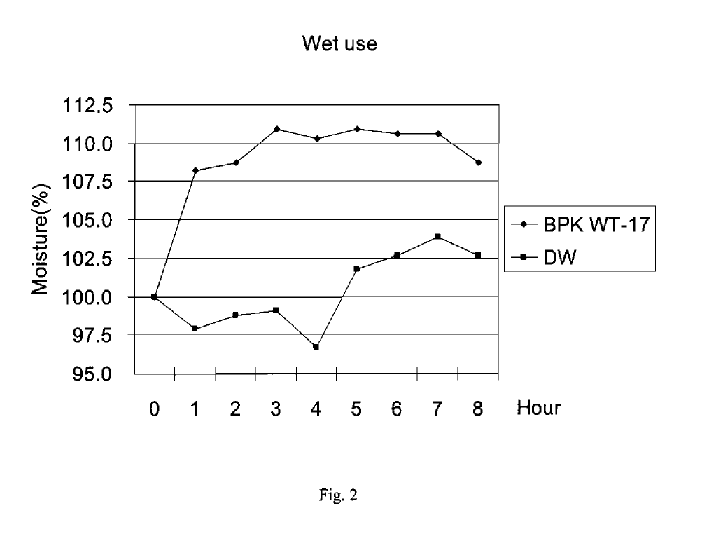

Part II of the study was conducted on the day after Part I was completed. The same panelists apply the foundations in the same manner as in Part I but using separate water dampened sponges. Each sponge is dampened by soaking in water and wringing well, leaving approximately 0.66 grams of water in the sponge. The results of Part II, shown in FIG. 2, are even more dramatic than the results of Part I. The composition of the present invention, when applied with a dampened sponge, not only results in greater skin moisturization over the 8 hour period as compared with the conventional powder foundation, but also boosts moisturization more than 10% as compared to the level of moisturization observed using the powder foundation of the present invention applied with a dry sponge.

### Example 3

### Lotion

TABLE 3

| Ingredients | Weight Percent |
|---|---|
| Sorbitol | 4.00 |
| Propylene glycol | 6.00 |
| PEG-32 | 5.00 |
| Oleth-20 | 0.50 |
| Methylcellulose | 0.20 |
| Caprylic/capric triglyceride | 0.70 |
| Trimethyldisilyl hydrolyzed silk | 0.30 |
| PG-propylmethlysilanediol crosspolymer | |
| Glycerin | 1.00 |
| Pentylene glycol | 2.00 |
| Butylene glycol | 0.20 |
| Tillandsia usneoides extract | 0.30 |
| Phenoxyethanol | 0.50 |
| Sodium citrate | 0.50 |
| Citric acid | 0.10 |
| Water | 78.70 |
| TOTAL | 100.00 |

### Example 4

### Essence (Serum)

TABLE 4

| Ingredients | Weight Percent |
|---|---|
| Stearyl alcohol | 0.50 |
| Squalane | 3.00 |
| Triethylhexanoin | 20.00 |
| Dipropylene glycol | 6.00 |
| PEG-8 | 4.00 |
| Caprylic/capric triglyceride | 0.70 |
| Trimethylsilyl hydrolyzed silk | 1.50 |
| PG-propylmethylsilanediol crosspolymer | |
| Glycerin | 5.00 |
| Pentylene glycol | 10.00 |
| Butylene glycol | 1.00 |
| Tillandsia usneoides extract | 1.50 |
| Sorbitan sesquiisostearate | 1.60 |
| Oleth-20 | 2.40 |
| Phenoxyethanol | 0.50 |
| Carbomer | 0.20 |
| Sodium hydroxide | 0.10 |
| Water | 42.00 |
| TOTAL | 100.00 |

### Example 5

### Cream

| Ingredients | Weight Percent |
|---|---|
| Stearyl alcohol | 6.00 |
| Squalane | 5.00 |
| Triethylhexanoin | 12.00 |
| Stearic acid | 2.00 |
| Dimethicone | 5.00 |
| PEG-32 | 6.00 |
| Caprylic/capric triglyceride | 0.70 |
| Trimethylsilyl hydrolyzed silk | 0.30 |
| PG-propylmethylsilanediol crosspolymer | |
| Glycerin | 1.00 |
| Pentylene glycol | 2.00 |
| Butylene glycol | 0.20 |
| Tillandsia usneoides extract | 0.30 |
| Ceteth-25 | 3.00 |
| Glyceryl stearate SE | 2.00 |
| Phenoxyethanol | 0.50 |
| Water | 54.00 |
| TOTAL | 100.00 |

### Example 6

### Cleansing Oil

| Ingredients | Weight Percent |
|---|---|
| Ethylhexyl palmitate | 52.90 |
| Squalane | 1.00 |
| Triethylhexanoin | 15.00 |
| Dimethicone | 5.00 |
| PEG-32 | 6.00 |
| Caprylic/capric triglyceride | 0.70 |
| Trimethylsilyl hydrolyzed silk | 0.30 |
| PG-propylmethylsilanediol crosspolymer | |
| Glycerin | 1.00 |
| Pentylene glycol | 2.00 |
| Butylene glycol | 0.20 |
| Tillandsia usneoides extract | 0.30 |
| Ceteth-25 | 10.00 |
| Oleth-20 | 5.00 |
| Phenoxyethanol | 0.50 |
| Water | 0.10 |
| TOTAL | 100.00 |

The foregoing description and examples illustrate selected embodiments of the instant invention. In light thereof variations and modifications will be suggested to one skilled in the art, all of which are within the spirit and purview of this invention.

What we claimed is:

1. A cosmetic composition providing a skin conditioning effect over extended periods of time when applied to skin in need of said skin conditioning, the composition comprising a skin conditioning sufficient amount of an extract of an air plant in combination with a cosmetically or dermatologically acceptable carrier.

2. The cosmetic composition of claim 1, wherein the extract is derived from the genus Tillandsia.

3. The cosmetic composition of claim 2, wherein the extract is derived from T. usneoides or T. recurvata.

4. The cosmetic composition of claim 1, which comprises in the range of from about 0.001 to about 10 weight percent, by weight of the total composition, of the air plant extract.

5. The cosmetic composition of claim 4, which comprises in the range of from about 0.01 to about 5 weight percent, by weight of the total composition, of the air plant extract.

6. The cosmetic composition of claim 1, wherein the skin conditioning effect is one or more of an emollient, skin protectant or humectant effect.

7. The cosmetic composition of claim 1, wherein the composition is in the form of a powder, a lotion, a cream, or a cleansing oil.

8. The cosmetic composition of claim 7, wherein the powder composition is an anhydrous powder composition.

9. The cosmetic composition of claim 8, which comprises in the range of from about 0.01 to about 5 weight percent of the air plant extract, and from about 80 to about 99 weight percent of a powder component, by total weight of the composition.

10. The cosmetic composition of claim 1, wherein the composition comprises an additional skin conditioning agent in an amount in the range of from about 0.001 to about 60 weight percent, by total weight of the composition.

11. The cosmetic composition of claim 9, wherein the composition comprises an additional skin conditioning agent in an amount in the range of from about 0.01 to about 15 weight percent, by total weight of the composition.

12. The cosmetic composition of claim 10, wherein the additional skin conditioning agent comprises one or more of caprylic/capric triglyceride, C9-15 fluoroalcohol phosphate, isotridecyl isononanoate, hyaluronic acid, glycerin, ethylhexylglycerin, urea, trehalose, pentylene glycol, butylene glycol, propylene glycol, sorbitol, hyaluronic acid, urea, and lactic acid.

13. The cosmetic composition of claim 11, wherein the additional skin conditioning agent comprises one or more of caprylic/capric triglyceride, C9-15 fluoroalcohol phosphate, isotridecyl isononanoate, hyaluronic acid, glycerin, ethylhexylglycerin, urea, trehalose, pentylene glycol, butylene glycol, propylene glycol, sorbitol, hyaluronic acid, urea, and lactic acid.

14. A method for providing a skin conditioning effect to skin over extended periods of time, comprising applying to the skin in need of said skin conditioning, a composition comprising a skin conditioning sufficient amount of an extract of air plant in combination with a cosmetically or dermatologically acceptable carrier.

15. The method of claim 14, wherein the extract of air plant is present in the composition in an amount in the range of about 0.001 to about 10 weight percent, by weight of the total composition.

16. The method of claim 14, wherein the extract is derived from Tillandsia usneoides or Tillandsia recurvata.

17. The method of claim 14, wherein the composition is in the form of a powder, a lotion, a cream or a cleansing oil.

18. The method of claim 17, wherein the powder is an anhydrous powder, and the effect is a skin moisturizing effect.

19. The method of claim 18, wherein the anhydrous powder composition comprises in the range of about 0.001 to about 10 weight percent of the air plant extract, and in the range of from about 80 to about 99 weight percent of a powder component present, by total weight of the composition.

20. The method of claim 19, wherein the powder composition comprises an additional skin conditioning agent in an amount in the range of from about 0.001 to about 15 weight percent, by total weight of the composition, and wherein the additional skin conditioning agent is selected from the group consisting of caprylic/capric triglyceride, C9-15 fluoroalcohol phosphate, isotridecyl isononanoate, hyaluronic acid, glycerin, ethylhexylglycerin, urea, trehalose, pentylene glycol, butylene glycol, propylene glycol, sorbitol, hyaluronic acid, urea, and lactic acid.
